(19) United States
(12) Patent Application Publication
(10) Pub. No.: US 2020/0179473 A1
(43) Pub. Date: Jun. 11, 2020

(54) ANTI-PATHOGENIC COMPOSITIONS

(71) Applicant: ANJON BIOLOGICS, INC., Las Vegas, NV (US)

(72) Inventor: Raziel RONEN, Hod Hasharon (IL)

(21) Appl. No.: 16/509,970

(22) Filed: Jul. 12, 2019

Related U.S. Application Data
(63) Continuation of application No. 15/557,749, filed on Sep. 12, 2017, now abandoned.
(60) Provisional application No. 62/134,840, filed on Mar. 18, 2015.

(51) Int. Cl. A61K 36/28; A61K 47/44; A61K 45/06; A61K 36/04; A61K 36/03

(52) U.S. Cl. CPC A61K 36/28; A61K 47/44; A61K 2300/00; A61K 36/04; A61K 36/03; A61K 45/06

(57) ABSTRACT

Antimicrobial, antiviral and antifungal composition and methods of use thereof are provided.

### FIELD OF THE INVENTION

The invention relates to, inter alia, compositions comprising Aaronsohnia factorovskyi extracts, and methods of use thereof, such as for treating viral and/or microbial infections.

### BACKGROUND

Many of the commonly used anti-bacterial, anti-viral and anti-fugal agents are synthetic compounds. In recent years, there has been an increased interest in developing and promoting the use of natural materials for use as anti-microbial, both in food preservation and in agricultural practices. One incentive for eliminating the use of synthetic compounds is emergence of anti-microbial drug resistance in human pathogens.

There is an unmet need for improved natural anti-pathogenic compositions effective against a broad-spectrum of pathogens.

### SUMMARY OF THE INVENTION

The present invention provides, in some embodiments, compositions comprising Aaronsohnia factorovskyi extracts alone or combined with various components including but not limited to additional vegetative extracts. In some embodiments, said compositions have antimicrobial activity, such as antiviral, antibacterial and antifungal activity.

According to one aspect, there is provided a composition comprising an Aaronsohnia factorovskyi extract, and one or more carriers and/or excipients. In some embodiments, the composition further comprises one or more extracts selected from the group consisting of: Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark and Neroli. In additional embodiments, the composition further comprises one or more extracts selected from the group consisting of: Citrus bergamia, Origanum, Eucaliptus, Rosmarinus officinalis and Melaleuca alternifolia.

According to another aspect, there is provided an article comprising the composition described herein.

According to another aspect, there is provided a method of inhibiting or reducing a formation of load of a microorganism and/or a formation of a biofilm or biofouling on and/or within an article, the method comprising incorporating or coating the composition of the invention on and/or within said article.

According to another aspect, there is provided a method of treating, preventing or ameliorating an infection or microbial-associated inflammatory condition in a subject in need thereof, the method comprising contacting or administering to the subject a therapeutically acceptable amount of the composition of the invention, thereby treating, preventing or ameliorating the infection or microbial-associated inflammatory condition in said subject.

According to another aspect, there is provided the composition of the invention, for use in treating an infection in a subject in need thereof.

According to another aspect, there is provided use of the composition of the invention, for preparation of a medicament for treating an infection in a subject in need thereof.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE FIGURES

FIGS. 1A-F show the effectiveness of a composition of the present invention against Acinetobacter baumannii, E. coli, Candida albicanse, and Pseudomonas aeruginosa after 24 h and 48 h. FIGS. 1C and 1F are respective controls.

FIG. 2 is a graph showing somatic cell count before and after treating calf mastitis with an embodiment of the composition of the invention.

### DETAILED DESCRIPTION OF THE INVENTION

The present invention provides, in some embodiments, compositions comprising Aaronsohnia factorovskyi extracts alone or combined with additional vegetative extracts. In some embodiments, said compositions have anti-pathogenic and anti-microbial activity.

The present invention is based, in part, on the surprising finding that a composition comprising Aaronsohnia factorovskyi extract exhibits increased antimicrobial activity. It has been further discovered that compositions comprising combinations of A. factorovskyi extract together with extracts from additional vegetative sources showed increased antimicrobial activity, such as in terms of activity and/or range of susceptible pathogens. In some embodiments, the activity of the combination disclosed herein is synergistic, i.e., its activity is more than the sum of the activity of each individual component.

In some embodiments, the present invention relates to crude extracts, fractions and/or isolated compounds derived from Aaronsohnia factorovskyi. In one embodiment, said extract is prepared from at least one plant material selected from the group consisting of: leaves, flowers, and seeds of Aaronsohnia factorovskyi. In some embodiments, said Aaronsohnia factorovskyi extract comprises at least one of Guaiazulene and azulon.

In some embodiments, the compositions of the invention comprise crude extracts, fractions and/or isolated compounds derived from Artemisia, including but not limited to extracts prepared from the leaves and/or seeds of Artemisia.

In some embodiments, the compositions of the invention comprise crude extracts, fractions and/or isolated compounds derived from Salvia officinalis (sage), including but not limited to extracts prepared from the leaves and/or seeds of Salvia officinalis.

In some embodiments, the compositions of the invention comprise crude extracts, fractions and/or isolated compounds derived from Citronella, including but not limited to extracts prepared from the leaves and/or seeds of Citronella.

In some embodiments, the compositions of the invention comprise crude extracts, fractions and/or isolated compounds derived from Geranium, including but not limited to extracts prepared from the leaves and/or seeds of Geranium.

In some embodiments, the compositions of the invention comprise crude extracts, fractions and/or isolated compounds derived from F. Spearmint, Lamiaceae (mentha), including but not limited to extracts prepared from the leaves and/or seeds of mentha.

In some embodiments, the compositions of the invention comprise crude extracts, fractions and/or isolated compounds derived from Thyme, including but not limited to extracts prepared from the leaves and/or seeds of Thyme.

In some embodiments, the compositions of the invention comprise crude extracts, fractions and/or isolated compounds derived from Illicium verum (star anise), including but not limited to extracts prepared from the leaves and/or seeds of Illicium verum.

In some embodiments, the compositions of the invention comprise crude extracts, fractions and/or isolated compounds derived from Ocimum basilicum (basil), including but not limited to extracts prepared from the leaves and/or seeds of Ocimum basilicum.

In some embodiments, the compositions of the invention comprise crude extracts, fractions and/or isolated compounds derived from Cymbopogon (lemongrass), including but not limited to extracts prepared from the leaves and/or seeds of Cymbopogon.

In some embodiments, the compositions of the invention comprise crude extracts, fractions and/or isolated compounds derived from Laurus nobilis (bay leaves), including but not limited to extracts prepared from the fruit and/or seeds of Laurus nobilis.

In some embodiments, the compositions of the invention comprise crude extracts, fractions and/or isolated compounds derived from Pimenta racemosa (Mill.), including but not limited to extracts prepared from the leaves, fruits and/or seeds of Pimenta racemosa.

In some embodiments, the compositions of the invention comprise crude extracts, fractions and/or isolated compounds derived from Carum carvi (caraway), including but not limited to extracts prepared from the leaves, fruits and/or seeds of Carum carvi.

In some embodiments, the compositions of the invention comprise crude extracts, fractions and/or isolated compounds derived from Cinnamomum, including but not limited to extracts prepared from the leaves and/or bark of Cinnamomum.

In some embodiments, the compositions of the invention comprise crude extracts, fractions and/or isolated compounds derived from Zeylanicum Nees (i.e., Ceylon cinnamon or Cinnamomum zeylanicum Blume), including but not limited to extracts prepared from the leaves, fruits and/or seeds of Zeylanicum Nees.

In some embodiments, the compositions of the invention comprise crude extracts, fractions and/or isolated compounds derived from Syzygium aromaticum, including but not limited to extracts prepared from the clove buds, leaves, fruits and/or seeds of Syzygium aromaticum.

In some embodiments, the compositions of the invention comprise crude extracts, fractions and/or isolated compounds derived from Pistacia lentiscus (mastic tree), including but not limited to extracts prepared from the bark and/or leaves of Pistacia lentiscus.

In some embodiments, the compositions of the invention comprise crude extracts, fractions and/or isolated compounds derived from Cymbopogon nardus (Rendle; F. poaceae), including but not limited to extracts prepared from the leaves of Cymbopogon nardus.

In some embodiments, the compositions of the invention comprise crude extracts, fractions and/or isolated compounds derived from F. rutaceae, including but not limited to extracts prepared from the peel of F. rutaceae.

In some embodiments, the compositions of the invention comprise crude extracts, fractions and/or isolated compounds derived from Eugenia caryophyllata thumb, including but not limited to extracts prepared from the seeds and/or fruits of Eugenia caryophyllata.

In some embodiments, the compositions of the invention comprise crude extracts, fractions and/or isolated compounds derived from Cuminum, including but not limited to extracts prepared from the seeds and/or leaves of Cuminum.

In some embodiments, the compositions of the invention comprise crude extracts, fractions and/or isolated compounds derived from Apium graveolens (celery), including but not limited to extracts prepared from the leaves, seeds and/or roots of Apium graveolens.

In some embodiments, the compositions of the invention comprise crude extracts, fractions and/or isolated compounds derived from Foeniculum vulgare (fennel), including but not limited to extracts prepared from the flower, seeds and/or leaves of Foeniculum vulgare.

In some embodiments, the compositions of the invention comprise crude extracts, fractions and/or isolated compounds derived from Myristica fragrans, including but not limited to extracts prepared from the seeds of Myristica fragrans.

In some embodiments, the compositions of the invention comprise crude extracts, fractions and/or isolated compounds derived from Melissa officinalis, including but not limited to extracts prepared from the leaves of Melissa officinalis.

In some embodiments, the compositions of the invention comprise crude extracts, fractions and/or isolated compounds derived from Lavender, including but not limited to extracts prepared from the leaves and/or seeds of Lavender.

In some embodiments, the compositions of the invention comprise crude extracts, fractions and/or isolated compounds derived from Sandelwood, including but not limited to extracts prepared from Sandelwood bark.

In some embodiments, the compositions of the invention comprise crude extracts, fractions and/or isolated compounds derived from Neroli, including but not limited to extracts prepared from the fruits and/or seeds of Neroli.

In some embodiments, the compositions of the invention comprise crude extracts, fractions and/or isolated compounds derived from Citrus bergamia, including but not limited to extracts prepared from the peel of Citrus bergamia.

In some embodiments, the compositions of the invention comprise crude extracts, fractions and/or isolated compounds derived from Origanum, including but not limited to extracts prepared from the leaves of Origanum.

In some embodiments, the compositions of the invention comprise crude extracts, fractions and/or isolated compounds derived from Eucaliptus, including but not limited to extracts prepared from the leaves and/or bark of Eucaliptus.

In some embodiments, the compositions of the invention comprise crude extracts, fractions and/or isolated compounds derived from Rosmarinus officinalis, including but not limited to extracts prepared from the leaves and/or flowers of Rosmarinus officinalis.

In some embodiments, the compositions of the invention comprise crude extracts, fractions and/or isolated compounds derived from Melaleuca alternifolia (tee tree), including but not limited to extracts prepared from the leaves and/or seeds of Melaleuca alternifolia, such as tea tree oil.

In some embodiments, the compositions of the invention comprise A. factorovskyi extract and one or more extracts selected from the group consisting of: Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark and Neroli.

In some embodiments, the present invention relates to crude extracts, fractions and/or isolated compounds derived from Aaronsohnia factorovskyi and chamomile, optionally with one or more extracts selected from the group consisting of: Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark and Neroli.

In some embodiments, the compositions of the invention comprise A. factorovskyi extract and one or more extracts selected from the group consisting of: Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark, Neroli, Citrus bergamia, Origanum, Eucaliptus, Rosmarinus officinalis and Melaleuca alternifolia.

In some embodiments, the compositions of the invention comprise A. factorovskyi extract and a plurality of extracts selected from the group consisting of: Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark, Neroli, Citrus bergamia, Origanum, Eucaliptus, Rosmarinus officinalis and Melaleuca alternifolia. In the context of this embodiment, the term plurality refers to at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 extracts, wherein each possibility represent a separate embodiment of the invention.

In some embodiments, the compositions of the invention comprise or consists of a plurality of extracts derived from Aaronsohnia factorovskyi, Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark and Neroli. In the context of this embodiment, the term plurality refers to at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 extracts, wherein each possibility represent a separate embodiment of the invention.

In some embodiments, the compositions of the invention comprise or consists of a plurality of extracts derived from Aaronsohnia factorovskyi, Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark, Neroli, Citrus bergamia, Origanum, Eucaliptus, Rosmarinus officinalis and Melaleuca alternifolia. In the context of this embodiment, the term plurality refers to at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 or at least 25 extracts, wherein each possibility represent a separate embodiment of the invention.

In some embodiments, the compositions of the invention comprise one or more extracts derived from the group consisting of: Salvia, Citrus bergamia, Origanum, Eucaliptus, Rosmarinus officinalis and Melaleuca alternifolia. In some embodiments, the compositions of the invention comprise Salvia, Citrus bergamia, Origanum, Eucaliptus, Rosmarinus officinalis and Melaleuca alternifolia. According to some embodiments, a composition comprising extracts selected from Salvia, Citrus bergamia, Origanum, Eucaliptus, Rosmarinus officinalis and Melaleuca alternifolia enables penetration of the compositions described herein through a pathogen's membrane. According to another embodiment, said composition provides an in-vivo anti-microbial effect of the compositions described herein.

In some embodiments, the compositions of the invention further comprise one or more crude extracts, fractions and/or isolated compounds selected from the group consisting of croton lechleri, Medicago sativa (alfalfa), Thymus serpyllum, myrrh, ginger, chamomile, Sesamum indicum or combination thereof. In some embodiments, the compositions of the invention further comprise one or more ingredients selected from the group consisting of citric acid, ascorbic acid, tocopherol, vitamin A or combination thereof.

In some embodiments, the compositions of the invention further comprise one or more algae extract selected from the group consisting of Rhodophyta (red algae), Phaeophyceae Laminaria (brown algae) and Porphyra, or combination thereof.

In some embodiments, the compositions of the invention further comprise one or more ingredients selected from the group consisting of starch, carob powder, glycerin, cellulose, or combination thereof.

In some embodiments, said carrier of the compositions of the invention is selected from the group consisting of vegetable oil, such as coconut oil, cottonseed oil, pine oil, safflower oil, linseed oil, palm oil, peanut oil or combination thereof. In some embodiments, said carrier is selected from the group consisting of gum arabic, guar gum, and locust bean gum, or combination thereof.

The crude extracts, fractions and/or isolated compounds provide antibacterial, antifungal and anti-parasitic (e.g., helminthic) activity. The extracts may be used in the prevention and treatment of bacterial, viral, fungal and/or parasitic (e.g., helminthic) infections in a subject.

Extraction Methods

Those skilled in the art will appreciate that there are a number of methods for preparing extracts from crude plant material. These methods include, among others, cutting, chopping, macerating and/or grinding raw or dried plant material and adding at least one solvent in order to obtain a plant extract. It will also be appreciated that the crude plant material may be fresh material or dry plant material.

As used herein the term “crude extract” refers to a preparation of a plant extract obtained by removing secondary metabolites from the plant material with the aid of a suitable solvent. This may be done, for example, by submerging the crude plant material in a suitable solvent, removing the solvent and consequently evaporating all or nearly all of the solvent. As used herein the term “purified extract” refers to an extract obtained by separating the constituent parts of a crude extract from each other.

In some embodiments, crude extract is prepared by combining of the plant material (e.g., A. factorovskyi) with an extracting material (e.g., ethanol, oil, or water). In one embodiment, about 1 Kg of plant material is combined with 10 L of an extracting material. In an embodiment wherein said extraction material is ethanol or oil, the extraction period lasts for about 1-20 days, about 2-18 days, 5-15 days, or about 10 days. In an embodiment wherein the extraction is water, the extraction period lasts for about 10 minutes in 100° C. Thereafter, the liquids may be filtered using 200 mesh filter. One skilled in the art will appreciate that additional extraction methods and filtering steps may be used.

A variety of plant parts may be used to arrive at the requisite extract. Suitable plant parts include roots, bulbs, tubers, leaves, basal leaves, stems, stem nodes, stem internodes, galls, stalks, woody parts, flowers, inflorescences, fruits, infructescences, seeds and combinations thereof. The plant part may be fresh, dried, frozen, or lyophilized. The plant part may be ground or pulverized into a plant material using a homogenizer, a blender, a mortar and pestle, a sonicator, or a similar apparatus.

The plant extract typically is prepared by contacting the plant material with a solvent for an appropriate period of time. Non-limiting examples of suitable alcohol solvents include methanol, ethanol, propanol, butanol, acetone, dichloromethane, chloroform, glycerine, hexane, ethyl acetate, propylene glycol, water or combinations thereof. The concentration of solvent that is contacted with the plant material may range from about 1% to about 100%. In embodiments in which ethanol is the solvent, the concentration of ethanol may range from about 1% to about 20%, from about 20% to about 40%, from about 40% to about 60%, from about 60% to about 80%, or from about 80% to about 100%.

Additional methods for preparing the extracts disclosed herein include but are not limited to the use of oil (e.g., herbal/botanical), glycerin, steam, decoction, pressure, acids and alkaline liquids, or combinations thereof.

The period of time the plant material is contacted with the solvent may range from about 1 hour to about 5 days. In various embodiments, the plant material may be contacted with the solvent for about 1-24 hours, for about 24-48 hrs, for about 48-72 hours, for about 72-96 hours, or for about 96-120 hours. Upon removal of the extract from the plant material, the plant material may be extracted one or more additional times with fresh alcohol solvent.

The solvent may be removed from the plant extract to form a dry plant extract. Those of skill in the art are familiar with suitable techniques to remove the alcohol solvent including, without limit, evaporation, distillation, and lyophillization.

In some embodiments, the composition of the invention may further comprise at least one pharmaceutically acceptable excipient. Non-limiting examples of suitable excipients include diluents, binders, fillers, buffering agents, pH modifying agents, disintegrants, dispersing agents, stabilizers, preservatives, and coloring agents. The amount and types of excipients may be selected according to principles known to one skilled in the art.

In one embodiment, the excipient may include at least one diluent. Non-limiting examples of suitable diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lacitol, mannitol, malitol, sorbitol, xylitol, maltodextrin, and trehalose.

In another embodiment, the excipient may comprise a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In another embodiment, the excipient may include filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may comprise a buffering agent. Representative examples of suitable buffering agents include, but are not limited to, MOPS, HEPES, TAPS, Bicine, Tricine, TES, PIPES, MES, Tris buffers or buffered saline salts (e.g., Tris buffered saline or phosphate buffered saline).

In various embodiments, the excipient may include a pH modifier. By way of non-limiting example, the pH modifying agent may be sodium carbonate or sodium bicarbonate.

In another alternate embodiment, the excipient may also include a preservative. Non-limiting examples of suitable preservatives include antioxidants, such as alpha-tocopherol or ascorbate.

In a further embodiment, the excipient may include a disintegrant. Suitable disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth.

In yet another embodiment, the excipient may include a dispersion enhancer. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In a further embodiment, the excipient may include a lubricant. Non-limiting examples of suitable lubricants include minerals such as talc or silica; and fats such as vegetable stearin, magnesium stearate or stearic acid.

In still another embodiment, it may be desirable to provide a coloring agent. Suitable color additives include, but are not limited to, food, drug and cosmetic colors, drug and cosmetic colors, or external drug and cosmetic colors.

In some embodiments, the compositions of the invention further comprise purified shellac. In some embodiments, the compositions of the invention further comprise a detergent such as a pH based detergent.

The weight fraction of the excipient(s) in the composition may be about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

In one embodiment, the compositions disclosed herein comprise 0.1-10 wt % of said Aaronsohnia factorovskyi extract. In another embodiment, the compositions disclosed herein comprise 0.1-5 wt % of said Aaronsohnia factorovskyi extract. In another embodiment, said composition is used for external treatments.

In another embodiment, the compositions disclosed herein include a content ranging from 0.2 to 20 wt % of said extracts. In another embodiment, the compositions disclosed herein include a content ranging from 0.5 to 20 wt % of said extracts. In another embodiment, said composition is used for in vivo treatment.

In another embodiment, the composition disclosed herein comprises a substantially equal amount of a first group of extracts and a second group of extracts, wherein the first group of extracts comprises extracts selected from the group consisting of: Aaronsohnia factorovskyi, Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Zeylanicum Nees, Clove bud, Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark and Neroli; and wherein the second group of extracts comprises extracts selected from the group consisting of: Salvia, Citrus bergamia, Origanum, Eucaliptus, Rosmarinus officinalis and Melaleuca alternifolia. In another embodiment, the ratio between the first group of extracts and the second group of extracts is ranges from 60:40 to 50:50, 70:30- to 60:40 or 80:20-70:30, respectively.

In another embodiment, there is provided a composition comprising one or more extracts derived from Salvia, Citrus bergamia, Origanum, Eucaliptus, Rosmarinus officinalis and Melaleuca alternifolia. According to some embodiments, said composition enables penetration of the compositions described herein through a pathogen's membrane. According to another embodiment, said composition provides an in-vivo anti-microbial effect of the compositions described herein.

The concentration of the extracts in the composition can and will vary depending on the requested activity, target and/or substrate type and whether the extract is provided alone (e.g. A. factorovskyi) or in combination with additional extracts. In general, the composition is diluted to receive a final concentration of about 0.1%-50, 0.1%-40, 0.1%-30, 0.1%-20%, 0.1%-15, 0.1%-10, or alternatively 0.1%-5. In one embodiment, higher concentrations are used for external use. In another embodiment, a concentration of 0.1%-20 is used for pharmaceutical use (e.g. oral, intravenous and the like).

In one embodiment, the composition comprises or consists of extracts derived from A. factorovsky, Salvia officinalis, Citronella, Geranium, Thyme, Star anise, Citric Acid and vegetable oil.

In another embodiment, the composition comprises or consists of extracts derived from A. factorovsky, Salvia officinalis, Citronella, Geranium, Thyme, Star anise, Citric Acid, vegetable oil, vegetable oil fatty acids and Gum Arabic.

In another embodiment, the composition comprises or consists of extracts derived from A. factorovsky, Salvia officinalis, Citronella, Geranium, Thyme, Star anise, Citric Acid, vegetable oil, Vegetable oil fatty acids, Gum Arabic, detergents, Mentha, Lemon Grass, Sandelwood bark and Tea Tree Oil.

In another embodiment, the composition comprises or consists of extracts derived from A. factorovsky, Salvia officinalis, Citronella, Geranium, Thyme, Star anise, Citric Acid, vegetable oil, Vegetable oil fatty acids, Gum Arabic, detergents, Mentha, Lemon Grass, Sandelwood bark, Tea Tree Oil, Thyme, Lemon Grass-Cymbopogon, Pimenta racemosa, Caraway, Citric Acid and Ascorbic Acid.

In another embodiment, the composition comprises or consists of extracts derived from A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass-Cymbopogon, Pimenta racemosa, Caraway, Citric Acid, Ascorbic Acid, Artemisia, Ocimum basilicum and Laurus nobilis.

In another embodiment, the composition comprises or consists of extracts derived from A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass-Cymbopogon, Pimenta racemosa, Caraway, Citric Acid, Ascorbic Acid, Artemisia, Ocimum basilicum, Laurus nobilis, Cinnamon bark, Ceylon, Cinnamomum, Zeylanicum Nees, Clove bud, Mastic tree, Cymbopogon nardus Rendle, Pine Oil, Ginger, llicium, Guar gum and Detergent.

In another embodiment, the composition comprises or consists of extracts derived from A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass (Cymbopogon), Pimenta racemosa, Caraway, Citric Acid, Ascorbic Acid, Artemisia, Ocimum basilicum, Laurus nobilis, Cinnamon bark, Ceylon, Cinnamomum, Zeylanicum Nees, Clove bud, Mastic tree, Cymbopogon nardus Rendle, Pine Oil, Ginger, llicium, Guar gum, Myrrh, Citrus peel, Eugenia caryophyllata Thunb and Cumin.

In another embodiment, the composition comprises or consists of extracts derived from A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass-Cymbopogon, Pimenta racemosa, Caraway, Citric Acid, Ascorbic Acid, Artemisia, Ocimum basilicum, Laurus nobilis, Cinnamon bark, Ceylon. Cinnamomum, Zeylanicum Nees, Clove bud, Mastic tree, Cymbopogon nardus Rendle, Pine Oil, Ginger, llicium, Guar gum, Myrrh, Citrus peel, Eugenia caryophyllata Thunb, Cumin, Shellac, Fennel, Myristica fragrans, Foeniculum vulgare Mill, Melissa, Lavender and Neroli.

In another embodiment, the composition comprises or consists of extracts derived from A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass-Cymbopogon, Pimenta racemosa, Caraway, Citric Acid, Ascorbic Acid, Artemisia, Ocimum basilicum, Laurus nobilis, Cinnamon bark, Ceylon. Cinnamomum, Zeylanicum Nees, Clove bud, Mastic tree, Cymbopogon nardus Rendle, Pine Oil, Ginger, llicium, Guar gum, Myrrh, Citrus peel, Eugenia caryophyllata Thunb, Cumin, Shellac, Fennel, Myristica fragrans, Foeniculum vulgare Mill, Melissa, Lavender and Neroli.

In another embodiment, the composition comprises or consists of extracts derived from A. factorovsky, Artemisia, Ocimum basilicum L Basil, Laurus nobilis L Bay, Cinnamon bark, Ceylon, Cinnamomum, Zeylanicum Nees, Clove bud, Mastic tree, Cymbopogon nardus Rendle, Pine Oil, Ginger, llicium, Guar gum, Myrrh, Citrus peel, Eugenia caryophyllata Thunb, Cumin, Fennel sweet flowers, Myristica fragrans, Foeniculum vulgare Mill, Melissa, Lavender, Sandelwood bark, Neroli, Tea Tree Oil, Vegetable oil fatty acids and Gum Arabic.

In another embodiment, the composition comprises or consists of extracts derived from A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass-Cymbopogon, Pimenta racemosa, Caraway, Citric Acid, Ascorbic Acid, Citric Acid, Ascorbic Acid, Artemisia, Ocimum basilicum, Laurus nobilis, Cinnamon bark, Ceylon, Cinnamomum, Zeylanicum Nees, Clove bud, Mastic tree, Cymbopogon nardus Rendle, Pine Oil, Ginger, llicium, Guar gum, Myrrh, Citrus peel, Eugenia caryophyllata Thunb, Cumin, Shellac, Fennel, Myristica fragrans, Foeniculum vulgare Mill, Melissa, Lavender, Neroli, Celery, Salvia officinalis, Bergamot peel, Origanum, Eucaliptus, Rosmarinus officinalis L, Tea Tree Oil, Carob powder, Locust bean gum, Cellulose, Glycerin, Gum Arabic and Guar gum.

In some embodiments, Aaronsohnia factorovskyi is extracted by the use of ethanol, IPA, oil, herbal powders, propylene glycol, polyethylene glycol, water (e.g., mineral water), acids, triglycerides, or combinations thereof. In some embodiments, Salvia officinalis, Citronella, Geranium, Thyme, Star anise, Citric acid and Vegetable oil can be independently, extracted by infusion, decoction, squeezing or a combination thereof. In some embodiments, mineral water, acids, Star anise, citric acid, Gum Arabic, detergents, ascrobic acid, Ginger, llicium, Guar gum, Citrus peel and Cumin can be independently, extracted by either mineral water, pressure, filtering, infusion, decoction, squeezing or a combination thereof.

In some embodiments, Shellac is extracted by the use of IPA. In some embodiments, Artemisia, Thyme, Caraway, Cinnamon, Clove bud, Mastic tree and Laurus nobilis L. can be independently, extracted by ethanol. In some embodiments, Salvia officinalis L, Citronella, Mentha, Ocimum basilicum L Basil, Lemon Grass-Cymbopogon, Caraway, Cymbopogon nardus Rendle, Celery, Myristica fragrans and Clove bud can be independently, extracted by oils and herbal powders.

In some embodiments, Cinnamon bark, Ceylon, Cinnamomum, Zeylanicum Nees, Clove bud, Mastic tree, Foeniculum vulgare Mill and Sandelwood bark can be independently, extracted by either triglycerides and polyethylene glycol or a combination thereof.

In some embodiments, Salvia officinalis L, Bergamot, Origanum, Eucaliptus, Rosmarinus officinalis L, Tea Tree Oil, Locust bean gum, cellulose, Glycerin, Gum Arabic and Guar gum can be independently, extracted using mineral salts.

In some embodiments, A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass (i.e., Cymbopogon), Pimenta racemosa, Caraway, Citric Acid, Ascorbic Acid, Citric Acid, Ascorbic Acid, Artemisia, Ocimum basilicum, Laurus nobilis, Cinnamon bark, Ceylon, Cinnamomum, Zeylanicum Nees, Clove bud, Mastic tree, Cymbopogon nardus Rendle, Pine Oil, Ginger, llicium, Guar gum, Myrrh, Citrus peel, Eugenia caryophyllata Thunb, Cumin, Shellac, Fennel, Myristica fragrans, Foeniculum vulgare Mill, Melissa, Lavender, Neroli, Celery, Sage (Salvia officinalis), Bergamot peel, Origanum, Eucaliptus, Rosmarinus officinalis L, Tea Tree Oil, Carob powder, Locust bean gum, Cellulose, Glycerin, Gum Arabic and Guar gum can be extracted by either pressure or filtering or a combination thereof.

### Pharmaceutical Compositions

As described herein the crude extracts, fractions and/or isolated compounds of the invention are suitable for oral, nasal, topical (including buccal and sublingual), rectal, vaginal, aerosol, intravenous, cutaneous or subcutaneous use on a subject. The subject may include a plant, a living animal, including a mammal such as a human.

The crude extracts, fractions and/or isolated compounds can be prepared in any desired delivery form known in the art of pharmaceuticals for example, the extract may be prepared as a tablet, capsule, tincture, powder, inhalant, syrup, spray, lozenge, solutions, gargles, colloidal dispersions, emulsions (oil-in-water or water-in-oil), suspensions, sprays, aerosol, granule and/or liquid. Other conventional formulations, including known carriers and additives, will be readily apparent to those skilled in the art.

In some embodiments, the pharmaceutical composition of the invention is formulated for aerosol administration, such as for administration by inhalation by a subject in need thereof.

The extracts, compounds and compositions of the invention are prepared so that they may be administered orally, dermally, parenterally, nasally, ophthalmically, sublingually, rectally or vaginally. Dermal administration includes topical application or transdermal administration. Parenteral administration includes intravenous, intraarticular, intramuscular, and subcutaneous injections, as well as use of infusion techniques. In some embodiments, the composition of the invention is administered by intranasal or intraoral administration, using appropriate solutions, such as nasal solutions or sprays, aerosols or inhalants. Nasal solutions are usually aqueous solutions designed to be administered to the nasal passages in drops or sprays. Typically, nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal and oral preparations for inhalation, aerosols and sprays are known and include, for example, antibiotics and antihistamines and are used for asthma prophylaxis. One or more compounds of the invention may be present in association with one or more non-toxic pharmaceutically acceptable ingredients to form the composition. These compositions can be prepared by applying known techniques in the art such as those taught in Remington—The Science and Practice of Pharmacy, 21st Edition (2005), Goodman & Gilman's The Pharmacological Basis of Therapeutics, 11th Edition (2005) and Ansel's Parmaceutical Dosage Forms and Drug Delivery Systems (8th Edition), edited by Allen et al., Lippincott Williams & Wilkins, (2005).

The crude extracts, fractions and/or isolated compounds may be formulated as a pharmaceutical composition, by methods know to those skilled in the art. Pharmaceutically acceptable ingredients may be used. The term “pharmaceutically acceptable” refers to properties and/or substances which are acceptable for administration to a subject from a pharmacological or toxicological point of view. Further “pharmaceutically acceptable” refers to factors such as formulation, stability, patient acceptance and bioavailability which will be known to a manufacturing pharmaceutical chemist from a physical/chemical point of view.

The “suitable forms” of the pharmaceutical composition may be combined with “pharmaceutically acceptable carriers” and other elements known in the art to produce tablets, capsules, tinctures, powers, inhalants and/or liquids. The pharmaceutical composition may further be combined with other ingredients which promote the absorption of the extract(s) into the body.

Anti-Pathogenic Use

In some embodiments, the invention provides methods of treating an infection or lowering the risk of infection in a subject, wherein the method comprises administering an effective amount of the composition described herein to a subject. In one embodiment, the infection is selected from the group consisting of fungal, bacterial, viral and/or parasitic infections. In another embodiment, the subject may be a mammal, such as a human. Alternatively, the subject may be from a botanic source, such as a plant or a tree.

Also provided herein are methods for inhibiting or reducing the formation of load of a microorganism and/or a formation of a biofilm or biofouling. In some embodiments said load of microorganism is maintained substantially reduced over a period of up to at least six months.

In some embodiments, the invention provides methods of inhibiting or reducing a formation of load of a microorganism and/or a formation of a biofilm or biofouling in water. In some embodiments, the invention provides methods of inhibiting or reducing a formation of load of a microorganism and/or a formation of a biofilm or biofouling in soil and/or sand.

In some embodiments, the invention provides methods of inhibiting or reducing a formation of load of a microorganism and/or a formation of a biofilm or biofouling on and/or within an article, the method comprising incorporating or coating the composition described herein on and/or within said article.

In some embodiments, the composition of the invention further comprises a substrate, wherein said composition is incorporated or coated on at least a portion of said substrate. In some embodiments, the substrate is or forms a part of an article. In some embodiments, said substrate comprises or is made of a polymer, wood, a metal, glass, carbon, a biopolymer and/or silicon.

According to some embodiments, there is provided an article comprising the composition of the invention.

In some embodiments of the compositions, article or methods described herein, said microorganism or pathogens are selected from the group consisting of: viruses, fungi, parasites, yeast, bacteria, and protozoa.

The dosage of any compositions of the present invention will vary depending on the symptoms, age and body weight of the subject/patient, the nature and severity of the disorder to be treated or prevented, the route of administration, and the form of the subject composition. Any of the subject compositions may be administered in a single dose or in divided doses. Dosages for the compositions of the present invention may be readily determined by techniques known to those of skill in the art or as taught herein. It will be appreciated that the crude extract, fraction, isolated compound and/or pharmaceutical composition comprising the crude extracts, fractions and/or isolated compounds may be used in applications for human, animal and/or veterinary products. Further due to the nature of the compounds of the present invention it will be appreciated that the subject may also be a non-human organism, such as a plant.

The term “preventing”, when used in relation to an infectious disease, or other medical disease or condition, is well understood in the art, and includes administration of a composition which reduces the frequency of, or delays the onset of, symptoms of a medical condition in a subject relative to a subject which does not receive the composition. Prevention of an infection includes, for example, reducing the number of diagnoses of the infection in a treated population versus an untreated control population, and/or delaying the onset of symptoms of the infection in a treated population versus an untreated control population.

The term “prophylactic or therapeutic” treatment is well known to those of skill in the art and includes administration to a subject of one or more of the subject compositions. If the composition is administered prior to clinical manifestation of the unwanted condition (e.g., disease or other unwanted state of the subject) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The term “treating” is recognized by those of skill in the art and refers to curing, as well as ameliorating at least one symptom of a condition or disorder

The use of the crude extracts, fractions, isolated compounds and/or pharmaceutical compositions containing the compound of the invention entails administration of an effective amount of the crude extract, fraction, isolated compound and/or pharmaceutical composition containing the compound to a subject in order to prevent or treat a condition.

The term “effective amount” or “effective dose” in the context of preventing or treating a condition refers to the administration of an amount of the active plant extract to an individual in need of treatment, either a single dose or several doses of the extract or pharmaceutical composition containing the extract, fraction and/or isolated compound. As will be appreciated by those of ordinary skill in this art, the effective amount of a composition may vary depending on such factors as the desired biological endpoint, the drug to be delivered, the composition of any additional active or inactive ingredients, the target tissue and several other factors. The precise time of administration and amount of any particular subject composition that will yield the most effective treatment in a given patient will depend upon the activity, pharmacokinetics, and bioavailability of the subject composition, physiological condition of the patient (including age, disease type and stage, general physical condition, responsiveness to a given dosage, sex and type of medication), route of administration, and other factors which are known to those in the art. The guidelines presented herein may be used to optimize the treatment, e.g., determining the optimum time and/or amount of administration, which will require no more than, routine experimentation consisting of monitoring the subject and adjusting the dosage and/or timing.

Toxicity and therapeutic efficacy of compositions of the invention may be determined by standard pharmaceutical procedures in cell cultures or experimental animals, such as by determining the LD_{S0} and the ED₅₀. Data obtained from the cell cultures and/or animal studies may be used to formulating a dosage range for use in humans. The dosage of any subject composition lies preferably within a range of circulating concentrations that include the ED₅₀ which has little or no toxicity. The dosage may vary within this range depending upon the dosage form employed and the route of administration utilized. For compositions of the present invention, the therapeutically effective dose may be estimated initially from cell culture assays.

The compounds obtained from an extract may be further purified and/or modified by means of synthetic organic chemistry methods which are well-known in the art. The compositions of the invention may also be produced by synthetic organic chemistry methods well-known in the art.

The invention also relates in part to a method of treating an infection in a subject comprising administering to a subject in need thereof a therapeutically effective amount of a compound or composition of the present invention.

It will be appreciated that the compounds of the present invention have antimicrobial and anti-parasitic (e.g., antihelminthic) activity. As used herein the term “antimicrobial” includes bacteria, fungi, protozoans and viruses.

The infection may be a bacterial infection caused by a bacteria selected from, but not limited to, the following genera Acinetobacter, Actinobacillus, Actinomycetes, Aeromonas, Bacillus, Bordetella, Borrelia, Brucella, Campylobacter, Chlamydia, Clostridium, Corynebacterium, Enterobacter, Enterococcus, Erwinia, Erysipelothnx, Escherichia, Francisella, Klebsiella, Haemophilus, Legionella, Leptospira, Listeria, Moraxella, Mycobacterium, Mycoplasma, Neisseria, Nocardia, Pasturella, Pseudomonas, Rickettsia, Salmonella, Shigella, Spirillum, Staphylococcus, Streptobacillus, Streptococcus, Streptomyces, Treponema, Vibrio, Yersinia and Xanthomonas. Specifically, the bacterial infection may be caused by a bacterium selected from the following species Escherichia coli, Enterococcus faecalis, Mycobacterium aurum, Mycobacterium bovis, Mycobacterium fortuitum, Mycobacterium smegmatis, Pseudomonas aeruginosa, Staphylococcus aureus and Salmonella typhi and/or Helicobacter pillory.

Alternatively, the infection may be a fungal infection caused by a fungus selected from, but not limited to, the group consisting of Alternaria, Aspergillus, Candida, Cercospora, Cladosporium, Colletotrichum, Cryptococcus, Diplodia, Fusarium, Guignardia, Monilinia, Penicillium, Phytophthora, Plasmopara, Podosphaera, Puccinia, Pythium, Rhizoctonia, Rhizopus, Sclerotinia, Sphaerotheca, Trichoderma, Venturia and Verticillium. Specifically, the fungal infection may be selected from the group of species consisting of Aspergillus fumigatus, Aspergillus niger, Aspergillus parasiticus, Aspergillus terreus, Aspergillus hennebergii, Aspergillus amsteloda.rni, Aspergillus flavus, Candida albicans, Colletotrichum gloeosporioides, Cryptococcus neoformans, Fusarium oxysporum, Fusarium culmorum, Fusarium javanicum, Fusarium merismoides, Cladosporium herbarum, Discula pinicola, Paecilomyces variotti, Sporidesmirum cladosporioides, Penicillium digitum, Penicillium expansum, Penicillium italicum, Penicillium janthinellum, Trichoderma harzianum, Trichosporium cheteromorphum, Rhizoctonia solani, Altemaria humicola, Altemaria tenuis, Penicillium brevi-compactum, Penicillium chrysogenum, Fusarium moniliforme, Fusarium poa, Penicillium ochro-chloron, Phialophora fastigiata, Verticillium marguandi, Leptographium lundbergii, Penicillium cyclopium and Pullularia pullulans

In a further embodiment, the infection may be a helminthic infection caused by a helminth selected from, but not limited to, the group consisting of Ascaris, Ancylostoma, Haemonchus, Trichostrongylos, Necator, Trichuris and Uncinaria.

According to another aspect, there is provided cosmetic products comprising the extracts or compositions of the invention. In some embodiments, the cosmetic products are in a form, such as creams, gels, powders, lotions, ointment, sunscreens, lipstick, body wash, foams, sprays, and/or herbal extracts.

Another aspect of the invention is the use of the extracts or compositions comprising the same to provide an antimicrobial effect to a patient in need thereof.

Another aspect of the invention is the use of the extracts or compositions comprising the same to provide an antimicrobial effect to a food or cosmetic composition.

Another aspect of the invention is the use of the extracts or compositions comprising the same to provide an antimicrobial effect to a surface. The effect may be produced by exposing the surface with the extracts or compositions of the invention or by laminating or embedding the extracts or compositions of the invention onto the surface itself. Another aspect of the invention are products with a surface which comprise the extracts or compositions of the invention which include, but are not limited to counter tops, doors, windows, handles, surgical equipment, medical tools, contact surfaces that can contaminate humans, animals, etc.

Another aspect of the invention is the use of the extracts or composition as an antimicrobial composition for coating a medical device. In one embodiment, the medical device is in the form of an implantable medical device. In another embodiment, the medical device is in the form of a fiber, mesh, powder, microspheres, flakes, sponge, foam, fabric, nonwoven, woven mat, a film, suture anchor device, suture, catheter, staple, stent, surgical tack, clips, plate and screw, drug delivery device, adhesion prevention barrier, and tissue adhesive.

Another aspect of the invention is the use of the extracts or composition as an antimicrobial agent in foods to improve preservation. In some embodiment, food preservation is the process of treating and handling food in a way that preserves its edibility and nutrition value.

Another aspect of the invention is the use of the extracts or composition as antimicrobial agents for the prevention and/or control of pre-harvest crop diseases comprising contacting an intended surface with the extracts or composition. In other embodiments, the extracts or composition are used as antimicrobial agents for the prevention and/or control of post-harvest rot in fruits and/or vegetables comprising contacting the fruits and/or vegetables with the extracts or composition. In other embodiments, the extracts or composition are used as antimicrobial agents for the prevention and/or control of post-harvest rot in fruits and/or vegetables and to prolong their shelf life (during the phases of storage, transport and sale), comprising contacting the fruits and/or vegetables with the extracts or composition.

Another aspect of the invention is the use of the extracts or composition as antimicrobial agents for treating animal diseases comprising administering the extracts or composition to an animal in need thereof. In other embodiments, the extracts or composition are used as antimicrobial agents for treating mastitis. In other embodiments, the extracts or composition are used as antimicrobial agents for treating gastrointestinal tract diseases.

Another aspect of the invention is the use of the extracts or composition as antimicrobial agents for treating infections in humans. In another embodiment, the extracts or compositions of the invention are used to treat a urinary tract infection (UTI). In another embodiment, the extracts or compositions of the invention are used to treat human stomach ulcers. In another embodiment, the extracts or compositions of the invention are used to treat human foot ulcers and/ or calluses. In another embodiment, the extracts or compositions of the invention are used to treat neuropathy. In another embodiment, the extracts or compositions of the invention are used to treat peripherial arterial disease (PAD). In another embodiment, the extracts or compositions of the invention are used as anti-viral gel. In another embodiment, the anti-viral gel is used to treat a viral disease caused by the herpes simplex virus. In another embodiment, the anti-viral gel is used to treat blisters and/or ulcers caused by herpes simplex virus.

Another aspect of the invention is the use of the extracts or composition as antimicrobial mouth rinses. In another embodiment, the antimicrobial mouth rinse reduces the bacterial count and inhibits the bacterial activity in dental plaque. In another embodiment, the extract or composition is sprayed into the mouth of a subject. In another embodiment, a gargling solution comprising the composition is used to treat microbial infections in a mouth of a subject. In another embodiment, the composition is useful in treating mouth ulcers (known as aphthous stomatitis).

Another aspect of the invention is the use of the extracts or composition as antimicrobial agent for treating an infection, including but not limited to an infected throat, such as by contacting (e.g. spraying, rinsing) the composition disclosed herein to the inflamed area, thereby treating the infected throat.

In some embodiments, there is provided pharmaceutical compositions comprising a therapeutically effective amount of the extract(s) as described herein and a pharmaceutically acceptable carrier.

As used herein, “pharmaceutically-acceptable” means that drugs, medicaments or inert ingredients which the term describes are suitable for use in contact with the tissues of humans and lower animals without undue toxicity, incompatibility, instability, irritation, allergic response, and the like, commensurate with a reasonable benefit/risk ratio. For example, the term “pharmaceutically acceptable” can mean approved by a regulatory agency of the Federal or a state government or listed in the U.S. Pharmacopeia or other generally recognized pharmacopeia for use in animals, and more particularly in humans.

As used herein, “safe and effective amount” means an amount of compound or composition sufficient to significantly induce a positive modification in the condition to be treated, but low enough to avoid serious side effects (at a reasonable benefit/risk ratio), within the scope of sound medical judgment. The safe and effective amount of the compound or composition will vary with the particular condition being treated, the age and physical condition of the patient being treated, the severity of the condition, the duration of the treatment, the nature of concurrent therapy, the specific compound or composition employed, the particular pharmaceutically-acceptable carrier utilized, and like factors within the knowledge and expertise of the attending physician.

If the topical pharmaceutical compositions of the present invention are formulated as an aerosol and applied to the skin as a spray-on, a propellant is added to a solution composition. A more complete disclosure of propellants useful herein can be found in Sagarin, Cosmetics Science and Technology, 2nd Edition, Vol. 2, pp. 443-465 (1972).

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

### EXAMPLES

### Materials and Methods

Plate Count Method Validation

Tests were conducted by Pacific BioLabs (PBL) according to the lab's standard operating procedures (SOPs). A suspension of challenged microorganisms (the composition tested and Salmonella enteric or Escherichia coli.) was prepared and maintained. Dilutions of the suspensions were prepared such that the resulting suspension levels contained approximately 1*10³ colony forming units (CFU) per milliliter (mL). Next, the suspension was diluted in D/E broth to achieve 1:10 product dilution. 1 mL and 0.1 mL of 1:10 dilution were placed into duplicate appropriately labeled petri dishes. Less than 100 colony forming unit (CFU) of each of the microorganism suspension was individually inoculated into the plates. In parallel two control plates were prepared by inoculating the same volume of the suspension dilution. A second set of phosphate buffer control was prepared for each organism to serve as neutralizing efficacy by inoculating the same volume of suspension dilution. Approximately 15-20 mL of molten TSA agar was then poured into plates. The plates were swirled to mix and allowed to harden, then were placed in a 30-35° C. incubator for not more than 24 hours. The plates were removed from the incubator and the number of colonies present on each plate was counted.

In Vitro Time Kill Assay

Tests were conducted by Pacific BioLabs (PBL) according to the lab's standard operating procedures (SOPs). A suspension of challenged microorganisms (Salmonella enteric and Escherichia coli) was prepared and maintained such that the resulting suspension levels contained approximately 1*10⁸ colony forming units (CFU) per mL. 2% solution comprising the tested compositions and 98% sterile water was prepared. 10 mL of the solution (comprising 2% of the composition) was placed into a sterile tube. 0.1 mL of the challenged organism was inoculated into the test sample container at a target inoculation level of 1*10⁶ CFU/mL. A duplicate plate count was performed on each inoculum immediately prior to inoculation. These were the positive control counts. The average of the two plates was used to calculate the log reduction at each time point for each microorganism. The inoculated test article was immediately vortexed following inoculation using a sterile instrument. A plate count was than performed on the inoculated sample after 30 seconds, 60 seconds and 5 minutes. The plate count was performed as follows: 1 mL of the inoculated test article placed into 9 mL of the neutralizing diluent validated for use in the efficacy study as described above under plate count method validation. This was the 10⁻¹ dilution. 1 mL of the 10⁻¹ dilution was placed into 9 mL of the neutralizing diluent. This was the 10⁻² dilution. This dilution scheme was continued through a 10⁻⁵ dilution. 1 mL of each dilution was placed into duplicate appropriately labeled petri dishes and poured with molten TSA agar. 1 mL of the neutralizing diluent was plated in duplicate with molten TSA agar for use as a negative control. All plates were allowed to harden then were incubated in 30-35° C. for not less than 48 hours. The plates were removed from the incubator and the number of colonies present on each plate was counted. The dilution that has 25-250 colonies was utilized for calculation purposes. The duplicate plates of each dilution were averaged for calculation purposes. The total number of survivor at each time point was then calculated by multiplying the average count obtained by the dilution factor. An overall log reduction for each test microorganism was calculated for each exposure time point using the initial inoculums titer.

Minimal Inhibitory Concentration Analyses

Tests were conducted by Pacific BioLabs (PBL) according to the lab's standard operating procedures (SOPs). A suspension of challenged microorganisms (Salmonella enteric and Escherichia coli) was prepared and maintained such that the resulting suspension levels contained approximately 1*10⁶ colony forming units (CFU) per mL. 2% solution comprising the tested compositions and 98% sterile water was prepared. The solution was diluted in Tryptic Soy Broth (TSB) to obtain 1:2, 1:4, 1:8 and 1:16 dilution of the 2% composition. A positive control tube and a negative control tube were prepared, each containing 1 mL TSB. 1 mL of the organism suspension containing approximately 1*10⁶ CFU per mL was added to each of the composition dilution tubes and the positive control tube. The negative control tube remained un-inoculated. The tubes were incubated in 30-35° C. for 18-24 hours then examined for growth. 1 mL from each from each test tube was transferred to 9 mL of DEB. This product dilution was immediately mixed using a vortex. Subsequently, 1 mL was transferred onto TSA. The plates were incubated for minimum 24 hours and then examined for growth. The slightest evidence of growth was recorded as a positive test sample. No growth was recorded as a negative test sample. The minimal inhibitory concentration was the highest product dilution that tests negative as detected by the results on the TSA plate.

General Cell Count

These test were conducted by the association of public health labs according to israeli's standard no 885/3. A concentration of 5% of the composition was tested in the presence of 10⁶ or 10⁷ Escherichia coli, the positive control contained 10⁶ Escherichia coli and water and a the negative control contained the 5% concentration of the composition. General plate count was performed.

### Effectiveness and Activity of the Compositions of the Invention

(i) Tree Rehabilitation and Restoration:

Compositions comprising extracts of Aaronsohnia factorovskyi were used to test their effectiveness for tree rehabilitation. These tests were conducted by Shelef Agro Lab and included different types of trees such as Eucaliptus, Citrus, Oak and other types of trees. The composition was first diluted in water to obtain a concentration of 0.5% -5% of the composition. For treating the trees, 5-10 liters of diluted composition were planted in the soil proximal to the trees, sprayed on the branches of the trees or a combination thereof.

Surprisingly, a significant portion of the trees rehabilitated within a time frame of 30 days. Within 30 days, new leaves and branches started to grow on treated trees. A person with skill in the art can recognize different this treatment can be applied on different types of trees.

Further experiments were set to test tree rehabilitation and restoration using a second composition comprising the above components as well as ingredients: Salvia officinalis, Geranium, Star anise. A significant portion of the trees rehabilitated within a time frame of 15 days.

Tests were designed to examine the biocide effectiveness of the composition in respect of the pine wood protection against the affect of various groups of wood coloring and mold fungi. 27 fungi species representing 3 different ecological systematic groups (9 micromycetic species each) were used as infectious agents. The first group (I) comprised Aspergillus niger, Aspergillus terreus, Altemari a humicola, Penicillium brevi-compactum, Penicillium chrysogenum, Fusarium moniliforme, Fusarium poa, Penicillium ochro-chloron and Phialophora fastigiata. The second group (II) comprised: Aspergillus hennebergii, Cladosporium herbarum, Fusarium javanicum, Fusarium merismoides, Discula pinicola, Paecilomyces variotti, Sporidesmirum cladosporioides, Trichosporium cheteromorphum and Verticillium marguandi. The third group (III) comprised: Aspergillus amsteloda.rni, Aspergillus flavus, Altemaria tenuis, Fusarium culmorum, Leptographium lundbergii, Penicillium cyclopium, Pullularia pullulans and Trichodenna harzianum.

Mean area of the fungi affection of the surface as well as the stage of fungi development were evaluated visually after 5, 10, 15 days. The growth data on the control specimens indicates the high level of infectious background that shows test result reliability in evaluating test specimens. Biotest species from the second ecological systemic group are aggressive for the pine wood. The wood of test and control specimens is mostly spored by Penecillium and Aspergillus. Test results reveal that antiseptic solutions 0.2% supplied by the customer has not shown 100% fungicidal activity in suppression of biotests. 0.5% antiseptic solution showed relatively high fungicidal activity, and evidently can be used for the further development of the preparation effectiveness addition of effective preservation antiseptic. Test results show that 0.5% solution can be used as a part of a mixed preparation in conditions of relatively low moisture capacity of wood. Results are summarized in table 1.

TABLE 1
Biocide effectiveness of the composition in respect of the pine wood protection
against the affect of various groups of wood coloring and mold fungi

| | | The composition of the invention | | | | | | | |
|---|---|---|---|---|---|---|---|---|---|
| | | 0.2% | | | | 0.5% | | | |
| | | Affection of inoculated and control specimens (% of affected area | | | | | | | |
| | | and fungi development stage in marks) | | | | | | | |
| Biotests | Exposure | % | | mark | | % | | Mark | |
| (groups) | (days) | test | control | test | control | test | control | test | control |
| I | 5 | 35 | 5 | 1 | 0 | 30 | 0 | 1 | 0 |
| | 10 | 75 | 20 | 3 | 2 | 70 | 10 | 2 | 0 |
| | 15 | 95 | 30 | 5 | 3 | 100 | 20 | 5 | 2 |
| II | 5 | 20 | 10 | 1 | 0 | 15 | 5 | 1 | 0 |
| | 10 | 45 | 30 | 2 | 2 | 30 | 15 | 3 | 1 |
| | 15 | 100 | 55 | 4 | 3 | 100 | 35 | 4 | 3 |
| III | 5 | 30 | 5 | 1 | 0 | 35 | 0 | 1 | 0 |
| | 10 | 60 | 15 | 3 | 2 | 50 | 10 | 3 | 1 |
| | 15 | 90 | 35 | 5 | 3 | 90 | 20 | 5 | 2 |
| Association | 5 | 30 | 15 | 2 | 0 | 35 | 0 | 2 | 0 |
| culture | 10 | 70 | 20 | 3 | 2 | 80 | 10 | 4 | 1 |
| (1 + 2 + 3) | 15 | 100 | 35 | 5 | 4 | 100 | 15 | 5 | 2 |

6-marj scale:
0 - completely clean specimens, absence of conidium's germs and colony development (visually and under the microscope);
1 - visually clean specimens, however small mycelium nidi in the form of spots are visible, spores are absent;
2 - superficial mycelium development in the form numerous spots, no spores;
3 - abundant mycelium overgrowth on the specimen surface, beginning of the spore formation;
4 - visual examination shows dense mycelium growth and spore formation;
5 - deep mycelium affection of the whole specimen area with intense spore formation.

(ii) Effectiveness Against Nematode:

A composition comprising extracts derived from A. factorovsky, Salvia officinalis, Citronella, Geranium, Thyme, Star anise, Citric Acid, Vegetable oil was tested for its effectiveness against nematode. This composition showed good results against nematode. The composition was used to treat soil with nematodes; the soil was tested 24 hours post treatment of the soil with irrigation water comprising 0.02% to 0.05% of the composition. The soil was sampled and 100 gram of treated soil and untreated soil were examined by Shelef Agro Lab. The result presented 92% decrease in nematode presence from the untreated soil sample to the treated soil. As summarized on table 2, the composition used was effective for treating nematodes.

TABLE 2
Nematodes in presence treated and untreated soil.

| Sample | Nematodes | Nematode |
|---|---|---|
| origin | presence | level from 1-10 |
| Untreated soil | Meloidogyne | 10 (very high) |
| Treated soil | Meloidogyne | 1 (low) |

(iii) Effectiveness Against Pathogenic Bacterium and Yeast:

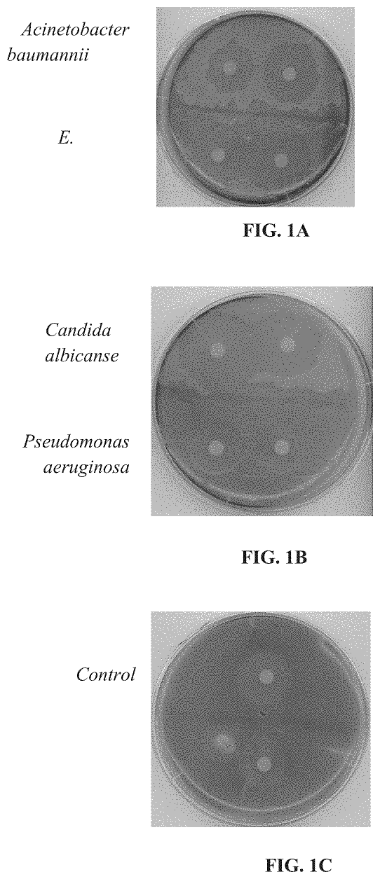

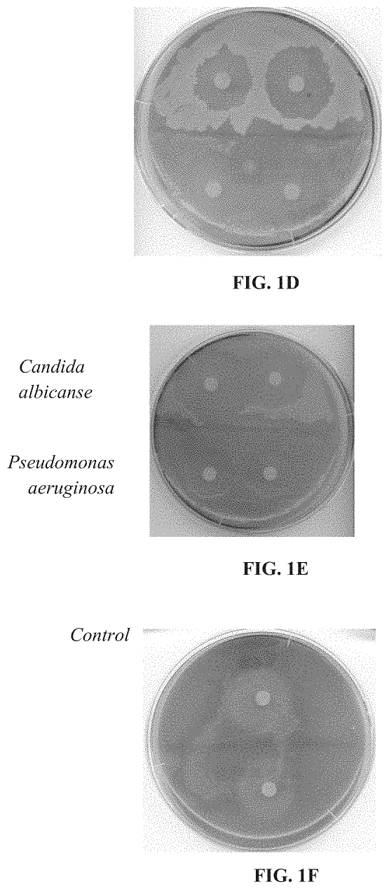

The antimicrobial effectiveness of a composition comprising extracts derived from A. factorovsky, Salvia officinalis, Citronella, Geranium, Thyme, Star anise, Citric Acid, Vegetable oil, Vegetable oil fatty acids, Gum Arabic, against bacterium and yeast was tested. FIGS. 1A-B show antibiogram results of the compositions described herein against Acinetobacter baumannii, E. coli, Candida albicanse, and Pseudomonas aeruginosa after 24 h and FIGS. 1D-E show the corresponding activity after 48 h. FIGS. 1C and 1F are respective controls.

(iv) Effectiveness as Poultry Hatcheries Disinfectant:

Currently, formalin or formaldehyde is used as poultry hatcheries disinfectant, such as for washing eggs, equipment and incubators. A composition comprising: A. factorovsky, Salvia officinalis, Citronella, Geranium, Thyme, Star anise, Citric Acid, Vegetable oil, Vegetable oil fatty acids, Gum Arabic, Detergents, Mentha, Lemon Grass, Sandelwood bark, Tea Tree Oil was examined and surprisingly shown to be effective as poultry hatchery disinfectant. Tests were conducted by Anjon Biologics. Table 3 summarizes the results of in-vitro time kill assay, demonstrating the effectiveness of the composition for treating bacterial and viral infections in human and animals.

TABLE 3
Evaluation of antimicrobial properties using in vitro time kill.

| Challenge | Product | CFU/mL of | Log 10 | Time to |
|---|---|---|---|---|
| organism | inoculation | sample | reduction | kill |
| S. enterica | 5.9*10⁵ | <1 | >5.8 | <30 seconds |
| E. coli | 7.1*10⁵ | <1 | >5.9 | <30 seconds |
| P. aeruginosa | 5.9*10⁵ | <1 | >5.8 | <30 seconds |
| Staphylococcus | 6.2*10⁵ | <1 | >5.9 | <30 seconds |
| aureus | | | | |
| Klebsiella | 6.8*10⁵ | <1 | >5.9 | <30 seconds |
| pneumoniae | | | | |

Resistant bacteria successfully treated with Scour Stopper in trials
Beta lactamases (EBSLs) enzyme producing E. coli
A clostridium difficile
Carbapenem-resistant Klebsiella pneumonia

(v) Effectiveness Preventing Animal Feed Oxidation Damage:

The effectiveness of a composition comprising extracts derived from A. factorovsky, Salvia officinalis, Citronella, Geranium, Thyme, Star anise, Citric Acid, Vegetable oil, Vegetable oil fatty acids, Gum Arabic, Detergents, Mentha, Lemon Grass, Sandelwood bark, Tea Tree Oil, Thyme, Lemon Grass (Cymbopogon), Pimenta racemosa, Caraway, Citric Acid, and Ascorbic Acid, for preventing animal feed oxidation damage was tested.

The feed oxidation test was conducted for 30 days, at 35° C. and 75% relative humidity. The performance of the composition of the invention was similar to that of known popular antioxidants in preventing animal feed oxidation damage.

(vi) Effectiveness Against Legionella pneumophila

Legionella pneumophila is a gram-negative bacterium found naturally in the environment, usually in water. Legionella pneumophila is a human pathogen that can cause Legionnaires disease.

The effectiveness of a composition comprising extracts derived from A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass-Cymbopogon, Pimenta racemosa, Caraway,Citric Acid, Ascorbic Acid, Citric Acid, Ascorbic Acid, Artemisia, Ocimum basilicum, Laurus nobilis, for treating Legionella pneumophila was examined.

The lab experiments were conducted by Hylabs Inc. Two portions of tap water were spiked with Legionella pneumophila. 1.5 gram of the composition was added to the test samples. Water samples were filtered through a microbiological membrane filter, and the two membranes were aseptically translated to two Hy Legionella Medium Selective B.C.Y.E. plates respectively and incubated as appropriate, next plate count was performed. Results summarized in Table 4, demonstrate that the composition inhibits the growth of Legionella pneumophila.

TABLE 4
Legionella pneumophila growth

| | Growth on reference | Growth on reference |
|---|---|---|
| | membrane | membrane |
| Inocula control | (water + inocula | (water + inocula + |
| Hy Legionella | without tested composition) | tested composition) |
| Medium Selective | Hy Legionella Medium | Hy Legionella Medium |
| B.C.Y.E. | Selective B.C.Y.E. | Selective B.C.Y.E. |
| 250 CFU | 128 CFU | 0 CFU |

In another experiment the composition was added to water pipes, water samples were tested for the presence of Legionella pneumophila prior and post treatment. Results are summarized in Table 5.

TABLE 5
Results of water examination

| | | Sampling | | | General | Remaining | Results | | |
|---|---|---|---|---|---|---|---|---|---|
| Specimen | Sampling | temperature | | Turbidity | Chlorine | chlorine | Lagionela | Lagionela | |
| description | time | ° C. | pH | NTU | ppm | ppm | CFU/1000 cc | identification | |
| (i) | Prior | 9:05 | 23.1 | 8.15 | 0.82 | 0.08 | 0.06 | <1 | |
| | treatment | | | | | | | | |
| | Post | 9:10 | 60.1 | 8.21 | 0.85 | 0.09 | 0.07 | <1 | |
| | treatment | | | | | | | | |
| (ii) | Prior | 9:15 | 21.2 | 8.22 | 0.7 | 0.1 | 0.08 | 2130 | Lp1 |
| | treatment | | | | | | | | |
| | Post | 9:20 | 58.4 | 8.18 | 0.62 | 0.06 | 0.05 | 8 | Lp1 |
| | treatment | | | | | | | | |
| (iii) | Prior | 9:25 | 31.1 | 8.2 | 0.72 | 0.09 | 0.07 | <1 | |
| | treatment | | | | | | | | |
| | Post | 9:30 | 59.1 | 8.22 | 0.75 | 0.1 | 0.08 | <1 | |
| | treatment | | | | | | | | |

(vii) Effectiveness Against Salmonella enteric and Escherichia coli Bacterium

The effectiveness of a composition comprising extracts derived from A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass-Cymbopogon, Pimenta racemosa, Caraway, Citric Acid, Ascorbic Acid, Citric Acid, Ascorbic Acid, Artemisia, Ocimum basilicum, Laurus nobilis, Cinnamon bark, Ceylon. Cinnamomum, Zeylanicum Nees, Clove bud, Mastic tree, Cymbopogon nardus Rendle, Pine Oil, Ginger, llicium, Guar gum, Detergent against two microorganism strains, Salmonella enteric and Escherichia coli was tested.

A time kill evaluation utilizing direct inoculation of the composition and determination of microbial survival was conducted as described in the materials and methods. The exposure time for evaluating microbial lethality were 30 seconds, 60 seconds or 5 minutes following inoculation. Results demonstrate the efficiency of the composition against Salmonella enteric and Escherichia coli. The total number of survivor at each time point for both Salmonella enteric and Escherichia coli was very low (<10). In addition the calculated overall log reduction for Salmonella enteric and Escherichia coli each exposure time point was >5.7 and >5 respectively (Table 6). A water solution comprising 2% of the composition was demonstrated to be sufficient to inhibit Salmonella enteric and Escherichia coli growth, as determined using the minimum Inhibitory concentration method described in the materials and methods. As shown in table 7 when further diluting the composition the inhibition effect is lost. The effect of the composition of the invention on Escherichia coli growth was also examined by general count of the number of colonies present on each plate using 5% composition and two concentrations of Escherichia coli by the association of public health the number of colonies present on each plate was counted (table 8).

TABLE 6
Minimal Inhibitory Concentration analyses

| | Salmonella enteric | | Escherichia coli | |
|---|---|---|---|---|
| | 5.4*10⁶ | | 1*10⁶ | |
| Organism | Average | | Average | |
| inoculum | Sample | Log10 | Sample | Log10 |
| Time point | CFU/mL | reduction | CFU/mL | reduction |
| 30 seconds | <10 | >5.7 | <10 | >5 |
| 60 seconds | <10 | >5.7 | <10 | >5 |
| 5 minutes | <10 | >5.7 | <10 | >5 |

TABLE 7
In vitro time kill assay

| Organism | Salmonella enteric | Escherichia coli |
|---|---|---|
| Inoculum | 5.4*10⁶ | 1*10⁶ |
| 1:1 undiluted water solution | No Growth | No Growth |
| comprising 2% of | | |
| the composition | | |
| 1:2 dilution | Growth | Growth |
| 1:4 dilution | Growth | Growth |
| 1:8 dilution | Growth | Growth |
| 1:16 dilution | Growth | Growth |
| Positive control | Growth | Growth |
| Negative control | No Growth | No Growth |

TABLE 8
General cell count

| | Result for 1 |
|---|---|
| Product | gram of product |
| 1 mL of 5% composition with 10⁷ E. coli | 0 |
| 1 mL of 5% composition with 10⁶ E. coli | 0 |
| 1 mL of 5% composition (negative control) | 0 |
| 1 mL water with 10⁶ E. coli | The plate is full |
| (positive control) | with colonies |

(viii) Effectiveness for Sand Sterilization and Biofilm Treatment

The effectiveness of an embodiment of the composition of the invention was tested for it use for sand sterilization and biofilm treatment. The tested composition comprised extracts derived from A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass-Cymbopogon, Pimenta racemosa, Caraway, Citric Acid, Ascorbic Acid, Artemisia, Ocimum basilicum, Laurus nobilis, Cinnamon bark, Ceylon. Cinnamomum, Zeylanicum Nees, Clove bud, Mastic tree, Cymbopogon nardus Rendle, Pine Oil, Ginger, llicium, Guar gum, Myrrh, Citrus peel, Eugenia caryophyllata Thunb and Cumin. Four square meters were treated with 8 liter of water comprising 400 mL of the composition (8% of the composition). Sand samples were collected prior and post treatment and analyzed in an independent lab (Bactochem) for the prescence of Escherichia coli. Results demonstrate a decrease from 560000 Escherichia coli per gram (MPN) prior treatment to 240 Escherichia coli per gram (MPN) post treatment.

(ix) Effectiveness for Treating Plant Diseases

Rhizopus is a genus of common saprobic fungi on plants and specialized parasites on animals. They are found on a wide variety of organic substrates, including fruits and vegetables. The effectiveness of a composition of the invention for treating Rhizopus in tomatoes was examined. The tested composition comprisesd extracts derived from A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass-Cymbopogon, Pimenta racemosa, Caraway, Citric Acid, Ascorbic Acid, Citric Acid, Ascorbic Acid, Artemisia, Ocimum basilicum, Laurus nobilis, Cinnamon bark, Ceylon. Cinnamomum, Zeylanicum Nees, Clove bud, Mastic tree, Cymbopogon nardus Rendle, Pine Oil, Ginger, llicium, Guar gum, Myrrh, Citrus peel, Eugenia caryophyllata Thunb, Cumin, Shellac, Fennel, Myristica fragrans, Foeniculum vulgare Mill, Melissa, Lavender and Neroli.

Leaves and tomatoes infected with Rhizopus fungi were treated with the composition for 9 days. The leaves and tomatoes as well as cropped tomatoes were each sprayed twice over nine days with a dilution comprising 0.5%-2% of the composition diluted in water or a control. A significant inhibition of fungal growth was observed after 5 days, resulting in healthy cropped tomatoes. A significant inhibition of fungal growth on the tomato plants was observed after 9 days, resulting in healthy tomatoes plants. A person with skill in the art can recognize the potential of the composition to treat other fruits and vegetables as well as other fungi species.

Phytophthora infestans is an oomycete that can infect potatoes, tomatoes and other members of the Solanaceae, it is considered a difficult disease to control today by ordinary methods. Tests were conducted in order to examine the effectiveness of the composition for treating Phytophthora infestans. Tomatoes plants infected with Phytophthora infestans were treated by spraying the plants with different dilutions comprising 0.05%, 0.2%, 0.3%, 0.4% of the compositions diluted in water or a control. Result after 7 days demonstrate the inhibition of Phytophthora infestans growth is best achieved by using 0.4% dilution of the composition, resulting in healthy plants.

(x) Effectiveness for Treating Mastitis

Mastitis in dairy cattle is the persistent, inflammatory reaction of the udder tissue. This potentially fatal mammary gland infection is a very common disease in dairy cattle. Mastitis can occur as a result of chemical, mechanical, or thermal injury or by invasion of bacteria. When mastitis is caused by bacterial infection, the bacterial toxins can damage the milk-secreting tissue, and various ducts throughout the mammary gland.

The effectiveness of a composition comprising extracts derived from A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass (Cymbopogon), Pimenta racemosa, Caraway, Citric Acid, Ascorbic Acid, Citric Acid, Ascorbic Acid, Artemisia, Ocimum basilicum, Laurus nobilis, Cinnamon bark, Ceylon. Cinnamomum, Zeylanicum Nees, Clove bud, Mastic tree, Cymbopogon nardus Rendle, Pine Oil, Ginger, llicium, Guar gum, Myrrh, Citrus peel, Eugenia caryophyllata Thunb, Cumin, Shellac, Fennel, Myristica fragrans, Foeniculum vulgare Mill, Melissa, Lavender and Neroli was examined as a treatment for cattle mastitis.

A first dose of 75 mL of the composition caused restimulation of the epithelia in the utters, and an undesired increase in somatic cells. Next, less components were used, a composition comprising extracts derived from A. factorovsky, Artemisia, Ocimum basilicum L Basil, Laurus nobilis L Bay leaves, fruits and seeds, Cinnamon bark, Ceylon. Cinnamomum leaves and bark, Zeylanicum Nees leaves, fruits and seeds, Clove bud leaves, fruits and seeds, Mastic tree bark, sapp and leaves, Cymbopogon nardus Rendle Leave extracts, Pine Oil, Ginger, llicium, Guar gum, Myrrh, Citrus peel, Eugenia caryophyllata Thunb, Cumin, Fennel sweet flowers, seeds and leaves, Myristica fragrans seeds Foeniculum vulgare Mill seeds, Melissa leaves, Lavender leaves and seeds, Sandelwood bark, Neroli seeds and fruit extract, Tea Tree Oil seeds and leaves, Vegetable oil fatty acids and Gum Arabic was found to decrease somatic cells.

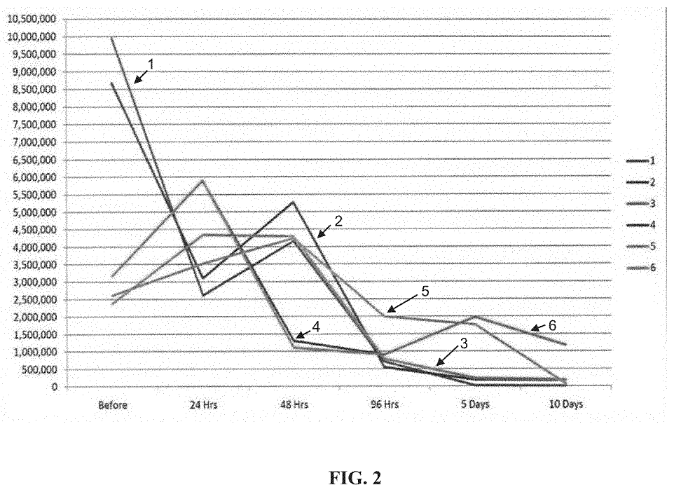

After laboratory testing of the composition, the composition was submitted for preliminary field tests to determine the efficacy and suitability of the treatment in field conditions. Tests were performed by Anjon Biologics. Infections ranged from subclinical infection to critical clinical disease levels. The three primary pathogens detected were S. enterica, Staphylococcus areus, Serratia and Escherichiac coli. Treatments were applied using intrammamary infusion. Applications were made using a single application of 30 mL. Significant reductions in the somatic cell counts (SCC) were achieved using the composition of the invention with one test case with a pre treatment SCC of 9,949,000 levels increased within 48 hours after treatment. Somatic cell counts were tested at 24, 48, 96 hours and 5 days and 10 days after treatment with six test cases showing significant reduction in somatic cell count as shown in FIG. 2.

(xi) Effectiveness for Treating Gastrointestinal Tract Disease

The therapeutic effectiveness of a composition comprising extracts derived from A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass-Cymbopogon, Pimenta racemosa, Caraway, Citric Acid, Ascorbic Acid, Artemisia, Ocimum basilicum, Laurus nobilis, Cinnamon bark, Ceylon, Cinnamomum, Zeylanicum Nees, Clove bud, Mastic tree, Cymbopogon nardus Rendle, Pine Oil, Ginger, llicium, Guar gum, Myrrh, Citrus peel, Eugenia caryophyllata Thunb, Cumin, Shellac, Fennel, Myristica fragrans, Foeniculum vulgare Mill, Melissa, Lavender, Neroli, Celery, Salvia officinalis, Bergamot peel extract, Origanum, Eucaliptus, Rosmarinus officinalis L, Tea Tree Oil, Carob powder, Locust bean gum, Cellulose, Glycerin, Gum Arabic, Gum Arabic and Guar gum, was demonstrated for treating gastrointestinal tract diseases in piglets and calves. The composition of the invention was used on calves with diverse stages of the gastrointestinal tract disease caused by Klebsiella infection. Results showed that within 24 hours of the first application of the composition of the invention, bowel discharge had returned to near normal with a medium brown color and mucous discharge has stopped. Calves showing signs of distress with progressive loose yellow discharge, that were still eating but not on regular basis, had returned to normal eating and drinking in 24-48 after the initial treatment. Calves with higher level of infection, showing thick mucus discharge, that stopped eating or drinking, returned to near normal bowel function over the 24-48 hours after the initial treatment. Intake of fluids started within 24-48 hours period with clearing of all symptoms within 3 days.

(xii) Effectiveness for Treating Human Stomach Ulcers Caused by Helicobacter pylori

Helicobacter pylori is a gram negative bacteria it is typically found in the epithelial cells underneath the mucus lining of the human stomach. Helicobacter pylori can cause ulcers in the lining of the stomach or the upper part of the small intestine and is also linked to the development of duodenal ulcers.

The efficiency of a composition comprising A. factorovsky, Salvia officinalis, Citronella, Geranium, Thyme, Star anise, Citric Acid, Vegetable oil, Vegetable oil fatty acids, Gum Arabic, Mentha, Lemon Grass, Sandelwood bark, Tea Tree Oil, Thyme, Lemon Grass-Cymbopogon, Pimenta racemosa, Caraway, Citric Acid, Ascorbic Acid, Citric Acid, Ascorbic Acid, Artemisia, Ocimum basilicum, Laurus nobilis, Cinnamon bark, Ceylon. Cinnamomum, Zeylanicum Nees, Clove bud, Mastic tree, Cymbopogon nardus Rendle, Pine Oil, Ginger, llicium, Guar gum, Myrrh, Citrus peel, Eugenia caryophyllata Thunb, Cumin, Shellac, Fennel, Myristica fragrans, Foeniculum vulgare Mill, Melissa, Lavender and Neroli, Celery leaves, seeds and roots, Sage-Salvia officinalis, Bergamot peel extract, Origanum leaves, Eucaliptus leaves and bark, Rosmarinus officinalis L leaves and flowers, Tea Tree Oil seeds and leaves, Carob powder, Locust bean gum, Cellulose, Glycerin, Gum Arabic, Gum Arabic, Guar gum for treating human stomach ulcers, was examined. Subjects suffering from pain related to stomach ulcers and/or duodenal ulcers were treated with the composition, using at least 5 drops of the composition in half a glass of water for 7-10 days. In the conclusion of 7-10 days of a daily treatment, tested subjects reported relief in symptoms including pain.

(xiii) Effectiveness for Treating Human Urinary Tract Infection

Urinary tract infection (UTI) is an infection that affects part of the urinary tract. UTI can be caused by Escherichia coli or other bacteria, viruses or fungi.

The efficiency of a composition of the invention for treating human urinary tract infection was examined. The tested composition comprised A. factorovsky, Artemisia, Salvia, Citronella, Geranium, Mentha, Thyme, Illicium verum (Star anise), Ocimum basilicum, Cymbopogon (Lemon Grass), Laurus nobilis, Pimenta racemosa, Carum carvi, Cinnamomum, Clove bud, Syzygium aromaticum, Pistacia lentiscus (Mastic tree), Cymbopogon nardus F. poaceae, F. rutaceae, Eugenia caryophyllata, Cuminum, Apium graveolens (Celery), Foeniculum vulgare (Fennel), Myristica fragrans, Melissa officinalis, Lavender, Sandelwood bark and Neroli.

Subjects were treated with the composition, using at least 5 drops of the composition in a glass of water per day for 3-7 days. Following 3 daily treatments, tested subjects reported relief in symptoms.

(xiv) Effectiveness for Treating Peripheral Arterial Disease (PAD), Neuropathy, Calluses and Foot Ulcers in Humans

Peripheral arterial disease is a circulatory problem in which narrowed arteries reduce blood flow to the foot/feet, resulting in oxygen deprivation of cells. This condition makes the skin more vulnerable to injury and slows the feet ability to heal. Peripheral neuropathy is nerve damage in the feet or lower legs, subjects suffering from peripheral neuropathy and/or peripheral arterial disease commonly suffer from open sores in the foot (foot ulcers) and toughened area of skin (calluses).

Subjects suffering from calluses and foot ulcers caused by peripheral arterial disease and/or Peripheral neuropathy were treated with a composition comprising A. factorovsky, Artemisia, Salvia, Citronella, Geranium, Mentha, Thyme, Illicium verum (Star anise), Ocimum basilicum, Cymbopogon (Lemon Grass), Laurus nobilis, Pimenta racemosa, Carum carvi, Cinnamomum, Clove bud, Syzygium aromaticum, Pistacia lentiscus (Mastic tree), Cymbopogon nardus F. poaceae, F. rutaceae, Eugenia caryophyllata, Cuminum, Apium graveolens (Celery), Foeniculum vulgare (Fennel), Myristica fragrans, Melissa officinalis, Lavender, Sandelwood bark and Neroli. In 5 to 7 weeks 90% of subjects show recovery of blood circulation, feet sensation and feet temperature and report better mobility of the legs. In addition the severity of foot ulcers and calluses were improved.

(xv) Effectiveness for Treating Herpes Simplex

Herpes simplex is a viral disease caused by the herpes simplex virus. Infections are categorized based on the part of the body infected. Oral herpes involves the face or mouth and may result in small blisters or may cause a sore throat. Genital herpes, often simply known as herpes, may have minimal symptoms or form blisters that break open and result in small ulcers.

A gel comprising a composition comprising A. factorovsky, Artemisia, Salvia, Citronella, Geranium, Mentha, Thyme, Illicium verum (Star anise), Ocimum basilicum, Cymbopogon (Lemon Grass), Laurus nobilis, Pimenta racemosa, Carum carvi, Cinnamomum, Clove bud, Syzygium aromaticum, Pistacia lentiscus (Mastic tree), Cymbopogon nardus F. poaceae, F. rutaceae, Eugenia caryophyllata, Cuminum, Apium graveolens (Celery), Foeniculum vulgare (Fennel), Myristica fragrans, Melissa officinalis, Lavender, Sandelwood bark and Neroli was found to be effective for treating herpes simplex virus.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims.

1. A composition comprising an extract of Aaronsohnia factorovskyi, and one or more carriers and/or excipients.

2. The composition of claim 1 comprising 0.1-10 wt % of said Aaronsohnia factorovskyi extract.

3. The composition of claim 1, further comprising one or more extracts selected from the group consisting of: Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Clove bud, Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark and Neroli.

4. The composition of claim 1, further comprising one or more extracts selected from the group consisting of: Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Clove bud Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark, Neroli, Citrus bergamia, Origanum, Eucaliptus, Rosmarinus officinalis and Melaleuca alternifolia.

5. The composition of claim 1, comprising an extract derived from Aaronsohnia factorovskyi and at least twenty extracts selected from the group consisting of: Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark, Neroli, Citrus bergamia, Origanum, Eucaliptus, Rosmarinus officinalis and Melaleuca alternifolia.

6. The composition of claim 1, comprising extracts derived from Aaronsohnia factorovskyi, Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark and Neroli.

7. The composition of claim 1, comprising extracts derived from Aaronsohnia factorovskyi, Artemisia, Salvia, Citronella, Geranium, Mentha, Thyme, Illicium verum, Ocimum basilicum, Cymbopogon, Laurus nobilis, Pimenta racemosa, Carum carvi, Cinnamomum, Syzygium aromaticum, Pistacia lentiscus, Cymbopogon nardus F. poaceae, F. rutaceae, Eugenia caryophyllata, Cuminum, Apium graveolens, Foeniculum vulgare, Myristica fragrans, Melissa officinalis, Lavender, Sandelwood bark, Neroli, Citrus bergamia, Origanum, Eucaliptus, Rosmarinus officinalis and Melaleuca alternifolia.

8. The composition of claim 2 including a content ranging from 0.2 to 20 wt % of said extracts.

9. The composition of claim 1, further comprising one or more ingredients selected from the group consisting of croton lechleri extract, Medicago sativa, Thymus serpyllum, myrrh, ginger, chamomile and Sesamum indicum, or combination thereof.

10. The composition of claim 1, further comprising one or more algae extract selected from the group consisting of Rhodophyta, Phaeophyceae Laminaria, and Porphyra, or combination thereof.

11. The composition of claim 1, wherein said carrier is selected from the group consisting of coconut oil, cottonseed oil, pine oil, safflower oil, linseed oil, palm oil, peanut oil, gum arabic, guar gum, and locust bean gum, or combination thereof.

12. The compositions of claim 1 for preventing or treating an infection.

13. The composition of claim 1, further comprising a substrate, wherein said composition is incorporated or coated on at least a portion of said substrate.

14. The composition of claim 12, wherein said substrate is or forms a part of an article.

15. The composition of claim 13, wherein said substrate comprises or is made of a polymer, wood, a metal, glass, carbon, a biopolymer and/or silicon.

16. An article comprising the composition of claim 1.

17. A method of inhibiting or reducing a formation of load of a microorganism and/or a formation of a biofilm or biofouling on and/or within an article, the method comprising incorporating or coating the composition of claim 1 on and/or within said article.

18. The method of claim 17, wherein said microorganism being selected from the group consisting of: viruses, fungi, parasites, yeast, bacteria, and protozoa.

19. The method of claim 17, wherein said load of microorganism is maintained substantially reduced over a period of up to at least six months.

20. A method of treating an infection in a subject in need thereof, the method comprising contacting or administering to the subject a therapeutically acceptable amount of the composition of claim 1.

21. A composition of claim 1, for use in treating an infection in a subject in need thereof.
